SEQUENCE LISTING (12 sequences)

SEQ ID NO: 1 - protein, 121 aa, Artificial Sequence
  huCD123-6Gv7 Heavy Chain Variable Region
QVQLVQSGAEVKKPGASVKVSCKASGYIFTSSIMHWVRQAPGQGLEWIGYIKPYNDGTKYNEKFKGRATLTSDRSTSTAYMELSSLRSEDTAVYYCAREGGNDYYDTMDYWGQGTLVTVSS

SEQ ID NO: 2 - protein, 108 aa, artificial sequence
  huCD123-6Gv4 Light Chain Variable Region
DIQMTQSPSSLSASVGDRVTITCRASQDINSYLSWFQQKPGKAPKTLIYRVNRLVDGVPSRFSGSGSGNDYTLTISSLQPEDFATYYCLQYDAFPYTFGQGTKVEIKR

SEQ ID NO: 3 - protein, 450 aa, artificial sequence
  huCD123-6Gv7-C442 Full Length Heavy Chain
QVQLVQSGAEVKKPGASVKVSCKASGYIFTSSIMHWVRQAPGQGLEWIGYIKPYNDGTKYNEKFKGRATLTSDRSTSTAYMELSSLRSEDTAVYYCAREGGNDYYDTMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPG

SEQ ID NO: 4 - protein, 214 aa, artificial sequence
  huCD123-6Gv4 Full Length Light Chain
DIQMTQSPSSLSASVGDRVTITCRASQDINSYLSWFQQKPGKAPKTLIYRVNRLVDGVPSRFSGSGSGNDYTLTISSLQPEDFATYYCLQYDAFPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 5 aa, artificial sequence
  huCD123-6Gv7-C442 Variable Heavy Chain CDR1
SSIMH

SEQ ID NO: 6 - protein, 17 aa, artificial sequence
  huCD123-6Gv7-C442 Variable Heavy Chain CDR2
YIKPYNDGTKYNEKFKG

SEQ ID NO: 7 - protein, 12 aa, artificial sequence
  huCD123-6Gv7-C442 Variable Heavy Chain CDR3
EGGNDYYDTMDY

SEQ ID NO: 8 - protein, 11 aa, artificial sequence
  huCD123-6Gv4 Variable Light Chain CDR1
RASQDINSYLS

SEQ ID NO: 9 - protein, 7 aa, artificial sequence
  huCD123-6Gv4 Variable Light Chain CDR2
RVNRLVD

SEQ ID NO: 10 - protein, 9 aa, artificial sequence
  huCD123-6Gv4 Variable Light Chain CDR3
LQYDAFPYT

SEQ ID NO: 11 - protein, 378 aa, Homo Sapiens
MVLLWLTLLLIALPCLLQTKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

SEQ ID NO: 12 - protein, 300 aa, Homo Sapiens
MVLLWLTLLLIALPCLLQTKEGGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

(19) United States
(12) Patent Application Publication
(10) Pub. No.: US 2019/0112359 A1
(43) Pub. Date: Apr. 18, 2019

(54) Separation of Triple-Light Chain Antibodies Using Cation Exchange Chromatography

(71) Applicant: ImmunoGen, Inc., Waltham, MA (US)

(72) Inventors: Fang LIU, Lexington, MA (US); Xinfang LI, Newton, MA (US)

(21) Appl. No.: 16/138,177

(22) Filed: Sep. 21, 2018

Related U.S. Application Data
(60) Provisional application No. 62/562,188, filed on Sep. 22, 2017.

(51) Int. Cl. C07K 16/06; C07K 16/24

(52) U.S. Cl. CPC C07K 16/065; C07K 16/244; C07K 2317/92; C07K 2317/51; C07K 2317/515; C07K 2317/565

(57) ABSTRACT

Methods of separating triple-light chain (H2L3) antibodies (e.g., anti-CD123 H2L3 antibodies) or antigen-binding fragments thereof from an antibody composition comprising H2L3 antibodies or antigen-binding fragments thereof and double-light chain (H2L2) antibodies (e.g., anti-CD123 H2L2) or antigen-binding fragments thereof are provided.

### CROSS-REFERENCE TO RELATED APPLICATION

This application claims the benefit of priority to U.S. Provisional Appl. No. 62/562,188, filed on Sep. 22, 2017, which is hereby incorporated by reference.

### REFERENCE TO SEQUENCE LISTING SUBMITTED ELECTRONICALLY

The content of the electronically submitted sequence listing (Name: 2921_0970001_ST25; Size: 15,734 bytes; and Date of Creation: Sep. 21, 2018) is herein incorporated by reference in its entirety.

### FIELD OF THE INVENTION

The field of the invention generally relates to methods of separating triple-light chain (H2L3) antibodies (e.g., anti-CD123 H2L3 antibodies) or antigen-binding fragments thereof from an antibody composition comprising H2L3 antibodies or antigen-binding fragments thereof and double-light chain (H2L2) antibodies (e.g., anti-CD123 H2L2) or antigen-binding fragments thereof.

### BACKGROUND

Recombinant antibodies engineered with reactive cysteine residues, i.e., “cysteine-engineered antibodies” can be covalently conjugated to drugs of interest to generate targeted therapeutics. Studies have shown that that mammalian cells stably transfected to express such cysteine engineered antibodies also secrete a high molecular weight species known as the triple light chain (H2L3) antibody (Gomez et al., Biotechnol Bioeng. 105(4): 748-60 (2010)). H2L3 cysteine-modified antibodies are the product of disulfide bond formation between an extra light chain and one of the engineered cysteine residues on a H2L2 cysteine-modified antibody. The level of H2L3 cysteine-modified antibodies in the cell culture is related to the cell line and the culture conditions. While the cell culture conditions can be modified to minimize H2L3 formation (e.g., by employing temperature shifts during cell culture), the impact is largely cell line dependent (Gomez et al., Biotechnol Prof 26(5): 1438-45 (2010)).

Due to the similarities to the monomer species, the separation of H2L3 antibodies generates a challenge during downstream purification of monoclonal H2L2 cysteine-modified antibodies. In one particular study, hydrophobic interaction chromatography (HIC) was found to reduce the H2L3 level from about 3% to 0.5% in purification of a non-cysteine engineered monoclonal antibody (Wollacott et al., mAbs 5(6): 925-935 (2013)). In the same study, cation exchange chromatography was used to attempt to remove H2L3 antibodies. However, even under modified conditions, this process was not sufficient to lower the percentage of H2L3 to less than 1% in all the cell lines tested. The authors concluded that the electrostatic effects in cation exchange chromatography are not strong enough to remove H2L3 antibodies. Thus, in order to achieve the most consistent antibody product (e.g., for therapeutic antibodies and immunoconjugates containing such antibodies), there is a need for a more effective separation of H2L3 species during antibody purification.

### BRIEF SUMMARY OF THE INVENTION

The present invention relates to the development of an effective purification strategy to separate triple light chain (H2L3) antibodies from double light chain (H2L2) antibodies. The methods take advantage of the fact that cation exchange resins separate proteins primarily based on charge. As provided herein, where the pH of the resin is lower than that of the antibody of interest (e.g., from 3.8 to 6.5), all antibody species, including both H2L3 and H2L2, bind to the cation exchange resin. When the antibodies are eluted from cation exchange resins using an elution composition with a high pH and/or a low salt concentration, the H2L3 species elute not only in late fractions after elution of the major peak of H2L2 species, but also in earlier fractions containing the desired H2L2 species. However, as demonstrated herein, optimization of cation exchange resins through the use of lower pH and higher salt concentrations can result in most or all of the H2L3 species eluting in late fractions after elution of the major peak of H2L2 species. Using optimized POROS™ XS strong cation exchange chromatography, the amount of H2L3 antibody in an antibody composition can be reduced from 11% to less than 1%, and this level of reduction is reproducible in various cell lines.

In some embodiments, a method of separating H2L3 antibodies or antigen-binding fragments thereof from an antibody composition comprising H2L3 antibodies or antigen-binding fragments thereof and H2L2 antibodies or antigen-binding fragments thereof comprises (i) applying the antibody composition to a cation exchange resin so that H2L3 antibodies or antigen-binding fragments thereof and H2L2 antibodies or antigen-binding fragments thereof bind to the resin; (ii) applying an elution composition with a pH of about 3.8 to about 5.0 to the cation exchange resin; and (iii) collecting an H2L2 composition eluted from the resin.

In some embodiments, a method of separating H2L3 antibodies or antigen-binding fragments thereof from an antibody composition comprising H2L3 antibodies or antigen-binding fragments thereof and H2L2 antibodies or antigen-binding fragments thereof comprises (i) applying the antibody composition to a cation exchange resin so that H2L3 antibodies or antigen-binding fragments thereof and H2L2 antibodies or antigen-binding fragments thereof bind to the resin; (ii) applying an elution composition with a salt concentration of about 300 mM to about 600 mM to the cation exchange resin; and (iii) collecting an H2L2 composition eluted from the resin.

In some embodiments, a method of separating anti-CD123 H2L3 antibodies or antigen-binding fragments thereof from an anti-CD123 antibody composition comprising anti-CD123 H2L3 antibodies or antigen-binding fragments thereof and anti-CD123 H2L2 antibodies or antigen-binding fragments thereof comprises (i) applying the anti-CD123 antibody composition to a cation exchange resin so that anti-CD123 H2L3 antibodies or antigen-binding fragments thereof and anti-CD123 H2L2 antibodies or antigen-binding fragments thereof bind to the resin; (ii) applying an elution composition with a pH of about 3.8 to about 5.5 to the cation exchange resin; and (iii) collecting an anti-CD123 H2L2 composition eluted from the resin, wherein the anti-CD123 H2L3 antibodies or antigen-binding fragments thereof and the anti-CD123 H2L2 antibodies or antigen-binding fragments thereof comprise the variable heavy chain CDR1, CDR2, and CDR3 sequences of SEQ ID NOs: 5-7, respectively and the variable light chain CDR1, CDR2, and CDR3 sequences of SEQ ID NOs: 8-10, respectively.

In some embodiments, a method of separating anti-CD123 H2L3 antibodies or antigen-binding fragments thereof from an anti-CD123 antibody composition comprising anti-CD123 H2L3 antibodies or antigen-binding fragments thereof and anti-CD123 H2L2 antibodies or antigen-binding fragments thereof comprises (i) applying the anti-CD123 antibody composition to a cation exchange resin so that anti-CD123 H2L3 antibodies or antigen-binding fragments thereof and anti-CD123 H2L2 antibodies or antigen-binding fragments thereof bind to the resin; (ii) applying an elution composition with a with a salt concentration of about 300 mM to about 600 mM to the cation exchange resin; (iii) and collecting an anti-CD123 H2L2 composition eluted from the resin, wherein the anti-CD123 H2L3 antibodies or antigen-binding fragments thereof and the anti-CD123 H2L2 antibodies or antigen-binding fragments thereof comprise the variable heavy chain CDR1, CDR2, and CDR3 sequences of SEQ ID NOs: 5-7, respectively and the variable light chain CDR1, CDR2, and CDR3 sequences of SEQ ID NOs: 8-10, respectively.

In some embodiments, no more than 2% of the antibodies or antigen binding fragments thereof in the H2L2 composition are H2L3 antibodies or antigen-binding fragments thereof. In some embodiments, no more than 1% of the antibodies or antigen binding fragments thereof in the H2L2 composition are H2L3 antibodies or antigen-binding fragments thereof. In some embodiments, no more than 0.5% of the antibodies or antigen binding fragments thereof in the H2L2 composition are H2L3 antibodies or antigen-binding fragments thereof.

In some embodiments, at least 98%, at least 99%, or at least 99.5% of the antibodies or antigen binding fragments thereof in the H2L2 composition are H2L2 antibodies or antigen-binding fragments thereof. In some embodiments, the H2L2 composition comprises no more than 25%, no more than 20%, no more than 15%, no more than 10%, or no more than 5% of the H2L3 antibodies or antigen-binding fragments thereof in the antibody composition applied to the cation exchange resin.

In some embodiments, the H2L2 composition comprises one or more eluted column volumes selected from column volumes 1-9. In some embodiments, the H2L2 composition comprises eluted column volumes 1-4. In some embodiments, the cation exchange resin comprises crosslinked poly(styrene divinylbenzene). In some embodiments, the cation exchange resin comprises a suflopropyl (—CH₂CH₂CH₂SO₃—) surface functionality. In some embodiments, the particle size of the cation exchange resin is about 50 μm. In some embodiments, the cation exchange resin has a bimodal pore size distribution. In some embodiments, the bimodal pore size distribution comprises pores about 500 nM in diameter and pores about 22 nM in diameter. In some embodiments, the cation exchange resin is a POROS™ Strong Cation Exchange Resin XS.

In some embodiments, the elution composition comprises a salt. In some embodiments, the salt in the elution composition is a chloride salt. In some embodiments, the chloride salt is sodium chloride, potassium chloride, calcium chloride, or magnesium chloride. In some embodiments, the concentration of salt in the elution composition is about 100 mM to about 600 mM, about 300 mM to about 500 mM, or about 350 mM to about 450 mM. In some embodiments, the concentration of salt in the elution composition is about 300 mM to about 500 mM, or about 350 mM to about 450 mM. In some embodiments, the concentration of salt in the elution composition is about 400 mM. In some embodiments, the elution composition has a pH of about 3.8 to about 6.5. In some embodiments, the elution composition has a pH of about 3.8 to about 5.0. In some embodiments, the elution composition has a pH of about 4.2.

In some embodiments, the method comprises applying an equilibration composition to the cation exchange resin prior to applying the antibody composition to the cation exchange resin. In some embodiments, the equilibration composition comprises sodium acetate. In some embodiments, the concentration of the sodium acetate in the equilibration composition is about 10 mM to 150 mM. In some embodiments, the concentration of the sodium acetate in the equilibration composition is about 50 mM. In some embodiments, the equilibration composition has a pH of about 3.8 to about 6.5. In some embodiments, the equilibration composition has a pH of about 4.2.

In some embodiments, the antibody composition comprises from about 10 to about 100 g/L protein. In some embodiments, the antibody composition comprises from about 30 g/L to about 50 g/L or from about 35 g/L to about 45 g/L protein. In some embodiments, the antibody composition comprises about 40 g/L protein. In some embodiments, the antibody composition has a pH of about 3.8 to about 6.5. In some embodiments, the antibody composition has a pH of about 4.2

In some embodiments, about 1% to about 20% of the antibodies or antigen-binding fragments thereof in the antibody composition are H2L3 antibodies or antigen-binding fragments thereof In some embodiments, about 1% to about 15%, or about 5% to about 15%, or about 3% to about 12%, or about 10% to about 15% of the antibodies or antigen-binding fragments thereof in the antibody composition are H2L3 antibodies or antigen-binding fragments thereof. In some embodiments, the H2L2 composition comprises at least 40%, at least 45%, at least 50%, or at least 55% of the H2L2 antibodies or antigen-binding fragments thereof in the antibody composition applied to the cation exchange resin.

In some embodiments, the antibody composition comprises cysteine-engineered antibodies or antigen-binding fragments thereof In some embodiments, the cysteine-engineered antibodies or antigen-binding fragments thereof comprise an engineered cysteine residue at EU/OU numbering position 442. In some embodiments, the antibody composition comprises antibodies. In some embodiments, the antibody composition comprises antigen-binding fragments of antibodies. In some embodiments, the antibody composition comprises a Fab, Fab′, F(ab′)₂, Fd, single chain Fv or scFv, disulfide linked Fv, V-NAR domain, IgNar, intrabody, IgGΔCH2, minibody, F(ab′)₃, tetrabody, triabody, diabody, single-domain antibody, DVD-Ig, Fcab, mAb², (scFv)₂, or scFv-Fc. In some embodiments, the antibody composition comprises antibodies or antigen-binding fragments thereof produced from a CHO cell line.

In some embodiments, the method further comprises conjugating the H2L2 antibodies or antigen-binding fragments thereof in the H2L2 composition to a cytotoxin to form an immunoconjugate composition. In some embodiments, the immunoconjugate composition is produced according to the methods described herein. In some embodiments, the immunoconjugate composition comprises no more than 2% H2L3 antibodies or antigen-binding fragments thereof. In some embodiments, the immunoconjugate composition comprises no more than 1% H2L3 antibodies or antigen-binding fragments thereof. In some embodiments, the immunoconjugate composition comprises no more than 0.5% H2L3 antibodies or antigen-binding fragments thereof.

In some embodiments, an H2L2 composition is produced according to the methods described herein. The H2L2 composition of claim 46 comprising no more than 2% H2L3 antibodies or antigen-binding fragments thereof. In some embodiments, the H2L2 composition comprises no more than 1% H2L3 antibodies or antigen-binding fragments thereof. In some embodiments, the H2L2 composition comprises no more than 0.5% H2L3 antibodies or antigen-binding fragments thereof.

In some embodiments, (i) the cation exchange resin comprises crosslinked poly(styrene divinylbenzene), a suflopropyl (—CH₂CH₂CH₂SO₃—) surface functionality, a particle size of about 50 μm, and a bimodal pore size distribution comprising pores about 500 nM in diameter and pores about 22 nM in diameter; (ii) the elution composition comprises about 300 to 600 mM of a chloride salt and a pH of about 3.8 to about 5.0; (iii) the antibody composition comprises from about 10 to about 100 g/L protein and about 10% to about 15% of the antibodies or antigen-binding fragments thereof in the antibody composition are H2L3 antibodies or antigen-binding fragments thereof; (iv) the H2L2 composition comprises one or more eluted column volumes selected from column volumes 1-9; and (v) no more than 2% of the antibodies or antigen binding fragments thereof in the H2L2 composition are H2L3 antibodies or antigen-binding fragments thereof.

In some embodiments, (i) the cation exchange resin comprises crosslinked poly(styrene divinylbenzene), a suflopropyl (—CH₂CH₂CH₂SO₃—) surface functionality, a particle size of about 50 μm, and a bimodal pore size distribution comprising pores about 500 nM in diameter and pores about 22 nM in diameter; (ii) the elution composition comprises about 400 mM NaCl and a pH of about 4.2; (iii) the antibody composition comprises from about 30 to about 50 g/L protein and about 10% to about 15% of the antibodies or antigen-binding fragments thereof in the antibody composition are H2L3 antibodies or antigen-binding fragments thereof; (iv) the H2L2 composition comprises eluted column volumes 1-4; and (v) no more than 1% of the antibodies or antigen binding fragments thereof in the H2L2 composition are H2L3 antibodies or antigen-binding fragments thereof.

In some embodiments, a composition comprises anti-CD123 antibodies or antigen-binding fragments thereof, wherein less than 1% of the anti-CD123 antibodies or antigen-binding fragments thereof are H2L3 antibodies or antigen-binding fragments thereof, and wherein the anti-CD123 antibodies or antigen-binding fragments thereof comprise the variable heavy chain CDR1, CDR2, and CDR3 sequences of SEQ ID NOs: 5-7, respectively and the variable light chain CDR1, CDR2, and CDR3 sequences of SEQ ID NOs: 8-10, respectively. In some embodiments, the anti-CD123 antibody comprises the variable heavy chain sequence of SEQ ID NO:1. In some embodiments, the anti-CD123 antibody or antigen-binding fragment thereof comprises the variable light chain sequence of SEQ ID NO:2. In some embodiments, the anti-CD123 antibody or antigen-binding fragment thereof is cysteine-engineered. In some embodiments, the anti-CD123 antibody comprises the heavy chain sequence of SEQ ID NO:3. In some embodiments, the anti-CD123 antibody comprises the light chain sequence of SEQ ID NO:4. In some embodiments, less than 0.5% of the anti-CD123 antibodies or antigen-binding fragments thereof are H2L3 antibodies or antigen-binding fragments thereof.

In some embodiments, a composition comprises anti-CD123 immunoconjugates, wherein the immunoconjugates comprise anti-CD123 antibodies or antigen-binding fragments thereof linked to DGN549-C , wherein less than 1% of the anti-CD123 antibodies or antigen-binding fragments thereof are H2L3 antibodies or antigen-binding fragments thereof, and wherein the anti-CD123 antibodies or antigen-binding fragments thereof comprise the variable heavy chain CDR1, CDR2, and CDR3 sequences of SEQ ID NOs: 5-7, respectively and the variable light chain CDR1, CDR2, and CDR3 sequences of SEQ ID NOs: 8-10, respectively. In some embodiments, the anti-CD123 antibody comprises the variable heavy chain sequence of SEQ ID NO:1. In some embodiments, the anti-CD123 antibody or antigen-binding fragment thereof comprises the variable light chain sequence of SEQ ID NO:2. In some embodiments, the anti-CD123 antibody or antigen-binding fragment thereof is cysteine-engineered. In some embodiments, the anti-CD123 antibody comprises the heavy chain sequence of SEQ ID NO:3. In some embodiments, the anti-CD123 antibody comprises the light chain sequence of SEQ ID NO:4. In some embodiments, the immunoconjugate has the following structure:

In some embodiments, less than 0.5% of the anti-CD123 antibodies or antigen-binding fragments thereof are H2L3 antibodies or antigen-binding fragments thereof.

### BRIEF DESCRIPTION OF THE DRAWINGS/FIGURES

FIG. 1 shows the size-exclusion ultra-performance liquid chromatography (SEC-UPLC) chromatogram of a cysteine engineered monoclonal antibody (CysmAb) composition following protein A purification. Designated peaks represent CysmAb monomer, aggregates, triple light chain antibody (H2L3), and low molecular weight (LMW) species. The y-axis of the chromatogram is a measure of the intensity of absorbance (in units of AU or Absorbance Units). The x-axis is in units of time (minutes), and is used determine the retention time for each peak.

FIG. 2 shows percentages of aggregates (dark gray bars) and H2L3 (light gray bars) antibodies in different bioreactor production batches (A, B, C, D, E, F, G, and H). Aggregates and H2L3 antibodies in batches A and B were produced in cell line A. Aggregates and H2L3 antibodies in batches C and D were produced in cell line B. Aggregates and H2L3 antibodies in batches E, F, G, and H were produced in cell line C.

FIG. 3 shows the percentage of H2L3 antibodies in the eluate after ceramic hydroxyapatite (CHT) purification under varying salt concentrations (100 mM, 95 mM, 90 mM, or 85 mM of potassium phosphate buffer). Load H2L3% (dark gray bars), Eluate H2L3% (light gray bars).

FIG. 4 shows the elution profile of the cysteine engineered mAb species overlaid on the elution profile of the aggregates and H2L3 species. The fraction yield % (black line), aggregates % (light gray bars), and H2L3% (dark gray bars) were measured for each of fractions 1-10 (F1-F10) (x-axis).

FIG. 5 shows the percentage of H2L3 in the elution pool at different elution pH. Higher pH (solid line, black circle), Medium pH (dashed line, open circle), Lower pH (dashed line, black circle), Starting H2L3 (straight black line).

FIG. 6 shows the percentage of H2L3 in the eluate at varying NaCl concentrations (420 mM, 410 mM, 400 mM, 390 mM, and 380 mM) and collection volumes (CV1-3, CV1-4, CV1-5, and less than/equal to 7 CV). Starting H2L3 (straight black line).

FIG. 7 shows the finalized elution conditions for POROS™ XS cation exchange chromatography based on statistical desirability analysis for purification of CysmAb.

FIG. 8A shows the chemical structure for IMGN632. IMGN632 is composition comprising immunoconjugates containing the anti-CD123 G4723 antibody linked to the cytotoxic payload DGN549-C in sodium bisulfite. The majority of the immunoconjugate in the composition is in the sulfonated version shown in FIG. 8A.

FIG. 8B shows an unsulfonated form of the immunoconjugate containing the anti-CD123 G4723 antibody linked to the cytotoxic payload DGN549-C (the mono-imine structure), which can also be present in an IMGN632 composition.

### DETAILED DESCRIPTION OF THE INVENTION

The present invention provides methods of separating H2L3 antibodies (e.g., anti-CD123 H2L3 antibodies) or antigen-binding fragments thereof from an antibody composition comprising H2L3 antibodies or antigen-binding fragments thereof and H2L2 antibodies (e.g., anti-CD123 antibodies) or antigen-binding fragments thereof.

### I. Definitions

To facilitate an understanding of the present invention, a number of terms and phrases are defined below.

A “cation exchange resin” refers to a solid phase which is negatively charged, and which has free cations for exchange with cations in an aqueous solution passed over or through the solid phase. Any negatively charged ligand attached to the solid phase suitable to form the cation exchange resin can be used, e.g., a carboxylate, sulfonate and others. Commercially available cation exchange resins include, but are not limited to, for example, those having a sulfonate based group; a sulfoethyl based group; a sulphopropyl based group; a sulfoisobutyl based; a sulfoxyethyl based group, a carboxymethyl based group; sulfonic and carboxylic acid based groups; a carboxylic acid based group; a sulfonic acid based group; and a orthophosphate based group. As provided herein, proteins (e.g., antibodies or antigen-binding fragments thereof) can be separated on the basis of the interaction between negatively charged groups in a cation exchange resin and positively charged groups on the proteins (e.g., antibodies or antigen-binding fragments thereof).

The term “elute,” and grammatical variations thereof, refers to the removal of a molecule, e.g., polypeptide of interest, from a resin (e.g., chromatography material) by using appropriate conditions, e.g., altering the ionic strength or pH of the buffer surrounding the resin (e.g., chromatography material), by altering the hydrophobicity of the molecule or by changing a chemical property of the ligand (e.g. charge), such that the protein of interest is unable to bind the resin and is therefore eluted from the resin (e.g., chromatography column). The term “eluate” refers to the effluent off the resin (e.g., column) containing the polypeptide of interest when the elution is applied onto the column. After elution of the polypeptide of interest, the resin (e.g., column) can be regenerated, sanitized and stored as needed.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

The term “antibody fragment” refers to a portion of an intact antibody with a sufficient positive charge to bind to a cation exchange resin. An “antigen-binding fragment” refers to a portion of an intact antibody that binds to an antigen and has a sufficient positive charge to bind to a cation exchange resin. An antigen-binding fragment can contain the antigenic determining variable regions of an intact antibody. Examples of antibody fragments include, but are not limited to Fab, Fab′, F(ab′)2, and Fv fragments, linear antibodies, and single chain antibodies.

The term “triple-light chain” or “H2L3” antibody or antigen-binding fragment refers to an antibody or antigen-binding fragment thereof that contains two heavy chains or fragments thereof and three light chains or fragments thereof.

The term “double-light chain” or “H2L2” antibody or antigen-binding fragment refers to an antibody or antigen-binding fragment thereof that contains two heavy chains or fragments thereof and two light chains or fragments thereof.

The term “antibody composition” refers to a composition comprising antibodies or antigen-binding fragments thereof. An antibody composition can comprise antibodies and other components that were produced in cell culture (e.g., from CHO cells), purified using a Protein A column, and optionally further purified using an anion exchange column. In addition to the antibodies or antigen-binding fragments thereof, an antibody composition may contain, for example, Tris acetic acid. An antibody composition can also contain aggregates.

An “H2L2” composition refers to a composition eluted from a cation exchange resin that contains a greater proportion of H2L2 species than the antibody composition applied to the cation exchange resin.

An “H2L3” composition refers to a composition eluted from a cation exchange resin that contains a greater proportion of H2L3 species than the antibody composition applied to the cation exchange resin.

The term “cysteine engineered” antibody or antigen-binding fragment thereof includes an antibody or antigen-binding fragment thereof with at least one cysteine (“Cys”) that is not normally present at a given residue of the antibody or antigen-binding fragment thereof light chain or heavy chain. Such Cys, which may also be referred to as “engineered Cys,” can be engineered using any conventional molecular biology or recombinant technology (e.g. , by replacing the coding sequence for a non-Cys residue at the target residue with a coding sequence for Cys). For example, if the original residue is Ser with a coding sequence of 5′-UCU-3′, the coding sequence can be mutated (e.g., by site-directed mutagenesis) to 5′-UGU-3′, which encodes Cys. In certain embodiments, the Cys engineered antibody or antigen-binding fragment thereof has an engineered Cys in the heavy chain. In certain embodiments, the engineered Cys is in or near the CH3 domain of the heavy chain. In certain embodiments, the engineered Cys is at residue 442 of the heavy chain (EU/OU numbering; EU index, Kabat et al, Sequences of Proteins of Immunological Interest, 5th Ed., NIH publication No. 91-3242, 1991, the entire contents of which are incorporated herein by reference). In certain embodiments, the Fc region comprises a cysteine at one or more of positions 239, 282, 289, 297, 312, 324, 330, 335, 337, 339, 356, 359, 361, 383, 384, 398, 400, 440, 422, and 442, as numbered by the EU index. In certain embodiments, any one or more of the following residues may be substituted with cysteine: V205 (Kabat numbering) of the light chain; A118 (EU numbering) of the heavy chain; and S400 (EU numbering) of the heavy chain Fc region. In certain embodiments, the variable light chain domain, e.g., of an scFv, has a cysteine at Kabat position 100. In certain embodiments, the variable heavy chain domain ,e.g. of an scFv, has a cysteine at Kabat position 44. Cysteine engineered antibodies may be generated as described, e.g., in U.S. Pat. Nos. 7,521,541, 7,855,275, U.S. Published Application No. 20110033378 and WO 2011/005481.

A “monoclonal” antibody or antigen-binding fragment thereof refers to a homogeneous antibody or antigen-binding fragment population involved in the highly specific recognition and binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal” antibody or antigen-binding fragment thereof refers to such antibodies and antigen-binding fragments thereof made in any number of manners including but not limited to by hybridoma, phage selection, recombinant expression, and transgenic animals.

The term “humanized” antibody or antigen-binding fragment thereof refers to forms of non-human (e.g. murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementary determining region (CDR) are replaced by residues from the CDR of a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some instances, the Fv framework region (FR) residues of a human immunoglobulin are replaced with the corresponding residues in an antibody or fragment from a non-human species that has the desired specificity, affinity, and capability. The humanized antibody or antigen-binding fragment thereof can be further modified by the substitution of additional residues either in the Fv framework region and/or within the replaced non-human residues to refine and optimize antibody or antigen-binding fragment thereof specificity, affinity, and/or capability. In general, the humanized antibody or antigen-binding fragment thereof will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody or antigen-binding fragment thereof can also comprise at least a portion of an immunoglobulin constant region or domain (Fc), typically that of a human immunoglobulin. Examples of methods used to generate humanized antibodies are described in U.S. Pat. 5,225,539; Roguska et al., Proc. Natl. Acad. Sci., USA, 91(3):969-973 (1994), and Roguska et al., Protein Eng. 9(10):895-904 (1996). In some embodiments, a “humanized antibody” is a resurfaced antibody.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al., Sequences of Proteins of Immunological Interest, (5th ed., 1991, National Institutes of Health, Bethesda Md.), “Kabat”); and (2) an approach based on crystallographic studies of antigen-antibody complexes (Al-lazikani et al, J. Molec. Biol. 273:927-948 (1997)). In addition, combinations of these two approaches are sometimes used in the art to determine CDRs.

The Kabat numbering system is generally used when referring to a residue in the variable domain (approximately residues 1-107 of the light chain and residues 1-113 of the heavy chain) (e.g., Kabat et al., Sequences of Immunological Interest. (5th Ed., 1991, National Institutes of Health, Bethesda, Md.) (“Kabat”).

The amino acid position numbering as in Kabat, refers to the numbering system used for heavy chain variable domains or light chain variable domains of the compilation of antibodies in Kabat et al. (Sequences of Immunological Interest. (5th Ed., 1991, National Institutes of Health, Bethesda, Md.), “Kabat”). Using this numbering system, the actual linear amino acid sequence can contain fewer or additional amino acids corresponding to a shortening of, or insertion into, a FR or CDR of the variable domain. For example, a heavy chain variable domain can include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues can be determined for a given antibody by alignment at regions of homology of the sequence of the antibody with a “standard” Kabat numbered sequence. Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |
| | | (Kabat Numbering) | |
| H1 | H31-H35 | H26-H35 | H26-H32 |
| | | (Chothia Numbering) | |
| H2 | H50-H65 | H50-H58 | H52-H56 |
| H3 | H95-H102 | H95-H102 | H95-H102 |

The term “human” antibody or antigen-binding fragment thereof means an antibody or antigen-binding fragment thereof produced by a human or an antibody or antigen-binding fragment thereof having an amino acid sequence corresponding to an antibody or antigen-binding fragment thereof produced by a human made using any technique known in the art. This definition of a human antibody or antigen-binding fragment thereof includes intact or full-length antibodies and fragments thereof.

The term “chimeric” antibodies or antigen-binding fragments thereof refers to antibodies or antigen-binding fragments thereof wherein the amino acid sequence is derived from two or more species. Typically, the variable region of both light and heavy chains corresponds to the variable region of antibodies or antigen-binding fragments thereof derived from one species of mammals (e.g. mouse, rat, rabbit, etc.) with the desired specificity, affinity, and capability while the constant regions are homologous to the sequences in antibodies or antigen-binding fragments thereof derived from another (usually human) to avoid eliciting an immune response in that species.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative embodiments are described in the following.

“Or better” when used herein to refer to binding affinity refers to a stronger binding between a molecule and its binding partner. “Or better” when used herein refers to a stronger binding, represented by a smaller numerical Kd value. For example, an antibody which has an affinity for an antigen of “0.6 nM or better”, the antibody's affinity for the antigen is <0.6 nM, i.e. 0.59 nM, 0.58 nM, 0.57 nM etc. or any value less than 0.6 nM.

By “specifically binds,” it is generally meant that an antibody binds to an epitope via its antigen binding domain, and that the binding entails some complementarity between the antigen binding domain and the epitope. According to this definition, an antibody is said to “specifically bind” to an epitope when it binds to that epitope, via its antigen binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

By “preferentially binds,” it is meant that the antibody specifically binds to an epitope more readily than it would bind to a related, similar, homologous, or analogous epitope. Thus, an antibody which “preferentially binds” to a given epitope would more likely bind to that epitope than to a related epitope, even though such an antibody may cross-react with the related epitope.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in certain embodiments, the polypeptides can occur as single chains or associated chains.

The term “immunoconjugate” or “conjugate” as used herein refers to a compound or a derivative thereof that is linked to a cell binding agent (i.e., an anti-CD123 antibody or fragment thereof) and is defined by a generic formula: C-A, wherein C=cytotoxin (e.g., a maytansinoid, a benzodiazepine compound, including pyrrolobenzodiazepines (PBD) and tetracyclic benzodiazepines, such as indolinobenzodiazepines) and A=antibody or antigen-binding fragment thereof, e.g., an anti-CD123 antibody or antibody fragment. An immunoconjugate can optionally contain a linker and be defined by the generic formula C-L-A, wherein C=cytotoxin, L=linker, and A=antibody or antigen-binding fragment thereof, e.g., an anti-CD123 antibody or antibody fragment. Immunoconjugates can also be defined by the generic formula in reverse order: C-A or A-L-C. Immunoconjugates can also contain multiple cytotoxins (C) per antibody or antigen-binding fragment thereof (A) or multiple cytotoxins (C) and linkers (L) per antibody or antigen-binding fragment thereof (A).

A “linker” is any chemical moiety that is capable of linking a compound, usually a drug (such as a maytansinoid, a benzodiazepine compound, including pyrrolobenzodiazepines (PBD) and tetracyclic benzodiazepines, such as indolinobenzodiazepines), to a cell-binding agent (such as an anti-CD123 antibody or a fragment thereof) in a stable, covalent manner. Linkers can be susceptible to or be substantially resistant to, e.g., disulfide bond cleavage, at conditions under which the compound or the antibody remains active. Suitable linkers are well known in the art and include, for example, disulfide groups and thioether groups.

The phrase “pharmaceutically acceptable” indicates that the substance or composition must be compatible chemically and/or toxicologically, with the other ingredients comprising a formulation, and/or the mammal being treated therewith.

The term “pharmaceutical formulation” refers to a preparation which is in such form as to permit the biological activity of the active ingredient to be effective, and which contains no additional components which are unacceptably toxic to a subject to which the formulation would be administered. The formulation can be sterile.

The terms “(human) IL-3Rα,” “Interleukine-3 Receptor alpha,” or “CD123,” as used interchangeably herein, refer to any native (human) IL-3Rα or CD123, unless otherwise indicated. The CD123 protein is an interleukin 3-specific subunit of a heterodimeric cytokine receptor (IL-3 Receptor, or IL-3R). The terms encompass “full-length,” unprocessed CD123 polypeptides as well as any form of CD123 polypeptide that results from processing within the cell. The term also encompasses naturally occurring variants of CD123, e.g., those encoded by splice variants and allelic variants. The CD123 polypeptides described herein can be isolated from a variety of sources, such as from human tissue types or from another source, or prepared by recombinant or synthetic methods. Where specifically indicated, “CD123” can be used to refer to a nucleic acid that encodes a CD123 polypeptide. Human CD123 sequences are known and include, for example, those sequences associated with NCBI reference numbers NP_002174 & NM_002183 (protein and nucleic acid sequences for human CD123 variant 1), and NP_001254642 & NM_001267713 (protein and nucleic acid sequences for human CD123 variant 2). As used herein, the term “human CD123” refers to CD123 comprising the sequence of SEQ ID NO:11 or SEQ ID NO:12.

| (SEQ ID NO: 11) | |
|---|---|
| 1 | MVLLWLTLLL IALPCLLQTK EDPNPPITNL RMKAKAQQLT WDLNRNVTDI ECVKDADYSM |
| 61 | PAVNNSYCQF GAISLCEVTN YTVRVANPPF STWILFPENS GKPWAGAENL TCWIHDVDFL |
| 121 | SCSWAVGPGA PADVQYDLYL NVANRRQQYE CLHYKTDAQG TRIGCRFDDI SRLSSGSQSS |
| 181 | HILVRGRSAA FGIPCTDKFV VFSQIEILTP PNMTAKCNKT HSFMHWKMRS HFNRKFRYEL |
| 241 | QIQKRMQPVI TEQVRDRTSF QLLNPGTYTV QIRARERVYE FLSAWSTPQR FECDQEEGAN |
| 301 | TRAWRTSLLI ALGTLLALVC VFVICRRYLV MQRLFPRIPH MKDPIGDSFQ NDKLVVWEAG |
| 361 | KAGLEECLVT EVQVVQKT |
| (SEQ ID NO: 12) | |
| 1 | MVLLWLTLLL IALPCLLQTK EGGKPWAGAE NLTCWIHDVD FLSCSWAVGP GAPADVQYDL |
| 61 | YLNVANRRQQ YECLHYKTDA QGTRIGCRFD DISRLSSGSQ SSHILVRGRS AAFGIPCTDK |
| 121 | FVVFSQIEIL TPPNMTAKCN KTHSFMHWKM RSHFNRKFRY ELQIQKRMQP VITEQVRDRT |
| 181 | SFQLLNPGTY TVQIRARERV YEFLSAWSTP QRFECDQEEG ANTRAWRTSL LIALGTLLAL |
| 241 | VCVFVICRRY LVMQRLFPRI PHMKDPIGDS FQNDKLVVWE AGKAGLEECL VTEVQVVQKT |

The term “anti-CD123 antibody” or “an antibody that binds to CD123” refers to an antibody that is capable of binding CD123 with sufficient affinity such that the antibody is useful as a diagnostic and/or therapeutic agent in targeting CD123 (e.g., the huMov19 (M9346A) antibody). The extent of binding of an anti-CD123 antibody to an unrelated, non-CD123 protein can be less than about 10% of the binding of the antibody to CD123 measured, e.g., by a radioimmunoassay (RIA).

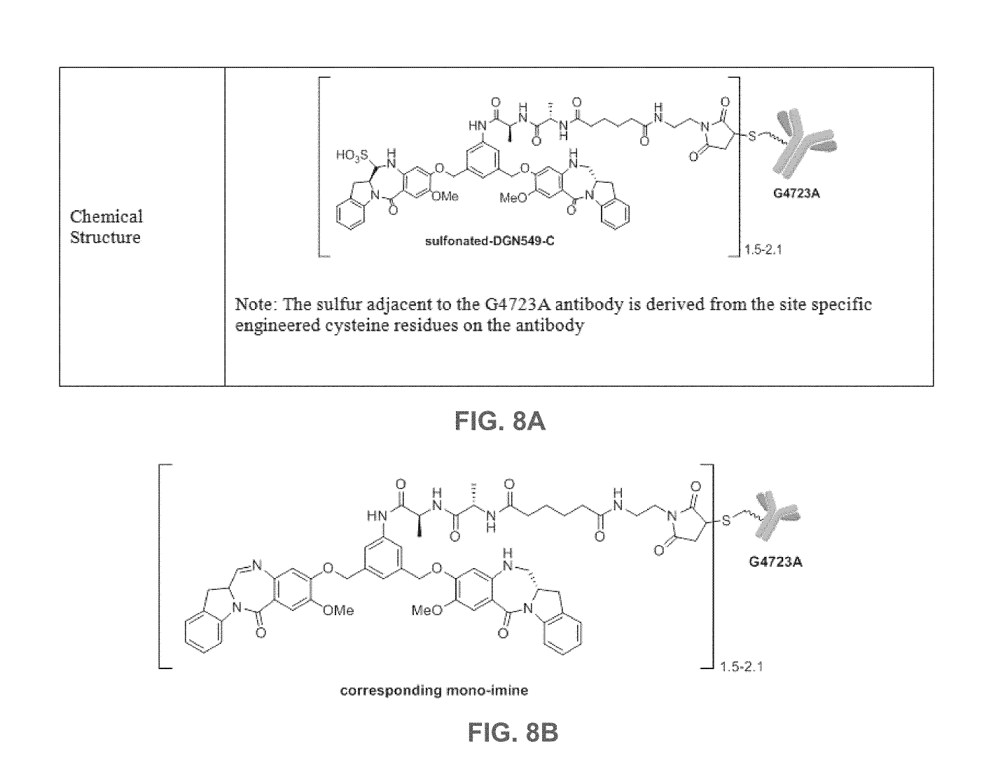

The term “IMGN632” refers to the immunoconjugate composition shown in FIG. 8. The immunoconjugate composition comprises immunoconjugates comprising an average of 1.5 to 2.1 DGN549-C cytotoxic agents per huCD123-6Gv4.7 (“G4723A”) antibody in a sulfonated version (FIG. 8A). The immunoconjugate composition can also comprise the unsulfonated immunoconjugate (the mono-imine structure shown in FIG. 8B).

As used in the present disclosure and claims, the singular forms “a,” “an,” and “the” include plural forms unless the context clearly dictates otherwise.

It is understood that wherever embodiments are described herein with the language “comprising,” otherwise analogous embodiments described in terms of “consisting of” and/or “consisting essentially of” are also provided.

The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. Cation Exchange Resins

According to the methods provided herein, cation exchange resins can be used to separate triple-light chain (H2L3) antibodies and antigen-binding fragments thereof from a composition comprising H2L3 and double-light chain (H2L2) antibodies and antigen-binding fragments thereof.

One exemplary cation exchange resin useful in the methods provided herein is an optimized POROS™ Strong Cation Exchange Resin XS (Thermos Fisher, previously Life Technologies Corporation, Carlsbad, Calif.; 10,000 mL=Cat. #440334; 5,000 mL=Cat. #4404335; 1,000 mL=Cat. #4404336; 250 mL=Cat. #4404337; 10 mL=Cat. # 82071, and 50 mL =Cat. #82072).

The cation exchange resin can comprise, for example, crosslinked poly(styrene divinylbenzene). The cation exchange resin can have a suflopropyl (—CH2CH2CH2SO3—) surface functionality. The cation exchange resin can comprise crosslinked poly(styrene divinylbenzene) and have a suflopropyl (—CH2CH2CH2SO3—) surface functionality.

In some embodiments, the cation exchange resin is not Fractogel SE HiCap (EMD Millipore) column. In some embodiments, the cation exchange resin is not methacrylate based.

The cation exchange resin can have a particle size of about 50 μm. The cation exchange resin can have a biomodal pore size distribution, e.g., with pores of about 500 nM in diameter and pores about 22 nM in diameter. The cation exchange resin can have a particle size of about 50 μm and a biomodal pore distribution with pores of 500 nM in diameter and pores about 22 nM in diameter.

The cation exchange resin can comprise crosslinked poly(styrene divinylbenzene), have a suflopropyl (—CH2CH2CH2SO3—) surface functionality, have a particle size of about 50 μm, and have a biomodal pore distribution with pores of 500 nM in diameter and pores about 22 nM in diameter.

The cation exchange resin can be of a particular size. For instance, the cation exchange resin can be about 10 to about 15,000 ml. The cation exchange resin can be about 20 to about 25 mL. The cation exchange resin can be about 100 to about 150 mL. The cation exchange resin can be about 10,000 to about 15,000 mL. The cation exchange resin can be about 13,800 mL. The cation exchange resin can be 32 L or greater.

The cation exchange resin can be a column.

### III. Antibodies and Antigen-Binding Fragments Thereof

Antibodies and antigen-binding fragments thereof (e.g., therapeutically useful antibodies and antigen-binding fragments thereof) generally contain two heavy chains or fragments thereof and two light chains or fragments thereof. However, triple-light chain (H2L3) species containing two heavy chains or fragments thereof and three light chains or fragments thereof have also been observed. Such H2L3 species can occur in higher rates in cysteine engineered antibodies and antigen-binding fragments thereof, for example where the H2L3 species is a result of a disulfide bond formed between an extra light chain and one of the engineered cysteines on the antibody or antigen-binding fragments thereof. Accordingly, an antibody composition as used herein can comprise cysteine-engineered antibodies or antigen-binding fragments thereof. Similarly, an H2L2 or H2L3 antibody or antigen-binding fragment thereof can be a cysteine-engineered H2L2 or H2L3 antibody or antigen-binding fragment thereof. The cysteine-engineered antibody or antigen-binding fragment thereof can, for example, comprise an engineered cysteine residue at EU/OU numbering position 442.

In some embodiments, the antibodies or antigen-binding fragments thereof are humanized antibodies or antigen-binding fragments thereof. In some embodiments, the humanized antibody or fragment is a resurfaced antibody or antigen-binding fragment thereof. In other embodiments, the antibodies or antigen-binding fragments thereof is a fully human antibody or antigen-binding fragment thereof.

By way of example, an anti-CD123 antibody or antigen-binding fragment thereof can be used in the present methods. The anti-CD123 antibody or antigen-binding fragment thereof can contain sequences of the huCD123-6Gv4.7 antibody shown below in Tables 1-3. For example, an anti-CD123 antibody or antigen-binding fragment thereof for use in the methods provided herein can comprise variable heavy chain CDR-1, CDR-2, and CDR-3 sequences of SEQ ID NOs: 5, 6, and 7, respectively and variable light chain CDR-1, CDR-2, and CDR-3 sequence of SEQ ID NOs: 8, 9, and 10, respectively. An anti-CD123 antibody or antigen-binding fragment thereof for use in the methods provided herein can comprise a variable heavy chain domain comprising the sequence set forth in SEQ ID NO:1. An anti-CD123 antibody or antigen-binding fragment thereof for use in the methods provided herein can comprise a variable light chain domain comprising the sequence set forth in SEQ ID NO:2. An anti-CD123 antibody or antigen-binding fragment thereof for use in the methods provided herein can comprise a variable heavy chain domain comprising the sequence set forth in SEQ ID NO:1 and a variable light chain domain comprising the sequence set forth in SEQ ID NO:2. An anti-CD123 antibody or antigen-binding fragment thereof for use in the methods provided herein can comprise a heavy chain comprising the sequence set forth in SEQ ID NO:3. An anti-CD123 antibody or antigen-binding fragment thereof for use in the methods provided herein can comprise a light chain comprising the sequence set forth in SEQ ID NO:4. An anti-CD123 antibody or antigen-binding fragment thereof for use in the methods provided herein can comprise a heavy chain comprising the sequence set forth in SEQ ID NO:3 and a light chain comprising the sequence set forth in SEQ ID NO:4.

In one example, an anti-CD123 antibody or antigen-binding fragment thereof for use in the methods provided herein can comprise a variable heavy chain domain and a variable light chain domain comprising the sequences set forth in Table 1. In another example, an anti-CD123 antibody or antigen-binding fragment thereof for use in the methods provided herein can comprise a heavy chain and the light chain comprising the sequences set forth in Table 2. In yet another example, an anti-CD123 antibody or antigen-binding fragment thereof for use in the methods provided herein can comprise variable heavy and light chain complementary determining regions comprising the sequences set forth in Table 3.

TABLE 1
huCD123-6Gv4.7 Heavy and Light Chain Variable
Regions

| Name | Sequence |
|---|---|
| huCD123-6Gv7 | QVQLVQSGAEVKKPGASVKVSCKASGYIFTSSIMH |
| Heavy Chain | WVRQAPGQGLEWIGYIKPYNDGTKYNEKFKGRA |
| Variable | TLTSDRSTSTAYMELSSLRSEDTAVYYCAREGGND |
| Region | YYDTMDYWGQGTLVTVSS |
| | (SEQ ID NO: 1) |
| huCD123-6Gv4 | DIQMTQSPSSLSASVGDRVTITCRASQDINSYLSWF |
| Light Chain | QQKPGKAPKTLIYRVNRLVDGVPSRFSGSGSGNDY |
| Variable | TLTISSLQPEDFATYYCLQYDAFPYTFGQGTKVEIK |
| Region | R |
| | (SEQ ID NO: 2) |

TABLE 2
huCD123-6Gv4.7-C442 Full Length Heavy and
Light Chain

| Name | Sequence |
|---|---|
| huCD123- | QVQLVQSGAEVKKPGASVKVSCKASGYIFTSSIMH |
| 6Gv7- | WVRQAPGQGLEWIGYIKPYNDGTKYNEKFKGRA |
| C442 | TLTSDRSTSTAYMELSSLRSEDTAVYYCAREGGND |
| Full | YYDTMDYWGQGTLVTVSSASTKGPSVFPLAPSSKS |
| Length | TSGGTAALGCLVKDYFPEPVTVSWNSGALTSGVHT |
| Heavy | FPAVLQSSGLYSLSSVVTVPSSSLGTQTYICNVNHKP |
| Chain | SNTKVDKKVEPKSCDKTHTCPPCPAPELLGGPSVFL |
| | FPPKPKDTLMISRTPEVTCVVVDVSHEDPEVKFNW |
| | YVDGVEVHNAKTKPREEQYNSTYRVVSVLTVLHQ |
| | DWLNGKEYKCKVSNKALPAPIEKTISKAKGQPREP |
| | QVYTLPPSRDELTKNQVSLTCLVKGFYPSDIAVEWE |
| | SNGQPENNYKTTPPVLDSDGSFFLYSKLTVDKSRW |
| | QQGNVFSCSVMHEALHNHYTQKSLCLSPG |
| | (SEQ ID NO: 3) |
| huCD123- | DIQMTQSPSSLSASVGDRVTITCRASQDINSYLSWF |
| 6Gv4 | QQKPGKAPKTLIYRVNRLVDGVPSRFSGSGSGNDY |
| Full | TLTISSLQPEDFATYYCLQYDAFPYTFGQGTKVEIK |
| Length | RTVAAPSVFIFPPSDEQLKSGTASVVCLLNNFYPRE |
| Light | AKVQWKVDNALQSGNSQESVTEQDSKDSTYSLSST |
| Chain | LTLSKADYEKHKVYACEVTHQGLSSPVTKSFNRGE |
| | C |
| | (SEQ ID NO: 4) |

TABLE 3
huCD123-6Gv4.7-C442 Variable Heavy and
Light Chain Complementary Determining
Regions

| Name | Sequence |
|---|---|
| huCD123-6Gv7-C442 | SSIMH |
| Variable | (SEQ ID NO: 5) |
| Heavy Chain CDR1 | |
| huCD123-6Gv7-C442 | YIKPYNDGTKYNEKFKG |
| Variable | (SEQ ID NO: 6) |
| Heavy Chain CDR2 | |
| huCD123-6Gv7-C442 | EGGNDYYDTMDY |
| Variable | (SEQ ID NO: 7) |
| Heavy Chain CDR3 | |
| huCD123-6Gv4 | RASQDINSYLS |
| Variable | (SEQ ID NO: 8) |
| Light Chain CDR1 | |
| huCD123-6Gv4 | RVNRLVD |
| Variable | (SEQ ID NO: 9) |
| Light Chain CDR2 | |
| huCD123-6Gv4 | LQYDAFPYT |
| Variable | (SEQ ID NO: 10) |
| Light Chain CDR3 | |

An anti-CD123 antibody or antigen-binding fragment thereof can bind to an epitope within amino acids 205 to 346 of human CD123.

An antibody or antigen-binding fragment thereof (e.g., a cysteine-engineered antibody or antigen-binding fragment thereof, an anti-CD123 antibody or antigen-binding fragment thereof, or a cysteine-engineered antibody or antigen-binding fragment thereof) for use in the present methods can be recombinantly produced. For example, an antibody or antigen-binding fragment thereof (e.g., a cysteine-engineered antibody or antigen-binding fragment thereof, an anti-CD123 antibody or antigen-binding fragment thereof, or a cysteine-engineered antibody or antigen-binding fragment thereof) for use in the present methods can be produced in a mammalian cell line, e.g., a CHO cell.

### IV. Antibody Compositions

According to the methods provided herein, antibody compositions comprising both triple-light chain (H2L3) antibodies and antigen-binding fragments thereof and double-light chain (H2L2) antibodies and antigen-binding fragments thereof can be applied to a cation exchange column to separate the H2L3 and H2L2 species.

The antibody compositions for use in the methods provided herein can be compositions in which about 1% to about 20% of the antibodies or antigen-binding fragments thereof are H2L3 antibodies or antigen-binding fragments thereof. The antibody compositions for use in the methods provided herein can be compositions in which about 1% to about 15%, or about 5% to about 15%, or about 3% to about 12%, or about 10% to about 15% of the antibodies or antigen-binding fragments thereof in the antibody composition are H2L3 antibodies or antigen-binding fragments thereof.

An antibody composition for use in the methods provided herein can comprise a particular protein concentration so that a particular loading density is applied to the cation exchange resin. The protein concentration (loading density) can be, for example, about 10 g/L to about 100 g/L. The protein concentration (loading density) can be about 30 g/L to about 50 g/L. The protein concentration (loading density) can be about 30 g/L to about 45 g/L. The protein concentration (loading density) can be about 30 g/L to about 40 g/L. The protein concentration (loading density) can be about 40 g/L.

In addition to H2L2 and H2L3 species, an antibody composition may contain aggregates. For example, an antibody composition can contain about 1 to about 10% aggregates. An antibody composition can contain about 1 to about 5% aggregates. An antibody composition can contain about 2 to about 5% aggregates.

The antibody composition can have a particular pH, e.g., about 3.8 to about 6.5. The antibody composition can have a pH of about 3.8 to about 5.5. The antibody composition can have a pH of about 3.8 to about 5.0. The antibody composition can have a pH of about 3.8 to about 4.7. The antibody composition can have a pH of about 3.8 to about 4.4. The antibody composition can have a pH of about 3.8 to about 4.2. The antibody composition can have a pH of about 4.0 to about 5.0. The antibody composition can have a pH of about 4.0 to about 4.7. The antibody composition can have a pH of about 4.0 to about 4.4. The antibody composition can have a pH of about 4.0 to about 4.2. The antibody composition can have a pH of about 4.2.

The pH of the antibody composition can be, for example, the same as the pH of an equilibration composition (binding composition), which, as described in more detail below, can be applied to the cation exchange resin before the antibody composition is applied to the cation exchange resin. The pH of the antibody composition can be, for example, the same as the pH of the elution composition, which, as described in more detail below, can be applied to the cation exchange resin after the antibody composition to elute an H2L2 composition. The pH of the antibody composition can be the same as the pH of the equilibration composition (binding composition) and the elution composition.

In some embodiments, the antibody composition does not have a pH of 6.0 In some embodiments, the antibody composition has a pH of less than 6.0.

An antibody composition can comprise protein A-purified antibodies or antigen-binding fragments thereof. The antibody composition can comprise antibodies or antigen-binding fragments thereof that have been protein-A purified and purified in an anion exchange column. Therefore, the antibody composition can contain components such as buffers (e.g., Tris acetic acid) and/or antibody aggregates, in addition to soluble H2L2 and H2L3 antibodies or antigen-binding fragments thereof.

### V. Elution Solutions and Methods of Eluting to Produce H2L2 and H2L3 Compositions

According to the methods provided herein triple-light chain (H2L3) antibodies and antigen-binding and double-light chain (H2L2) antibodies and antigen-binding fragments thereof can be separately eluted from a cation exchange column.

In particular, an elution composition can be applied to a cation exchange resin (e.g., column) to preferentially elute H2L2 species, and an H2L2 composition can then be collected from the resin. Elution compositions for use in such methods are provided herein.

An elution composition for use in the methods provided herein can comprise a salt. The salt can be as chloride salt, for example sodium chloride, potassium chloride, calcium chloride, or magnesium chloride. In one instance, the salt is sodium chloride. The concentration of the salt (e.g., sodium chloride) in the elution composition can be, for example about 100 mM to about 600 mM. The concentration of the salt (e.g., sodium chloride) in the elution composition can be about 200 mM to about 600 mM. The concentration of the salt (e.g., sodium chloride) in the elution composition can be about 300 mM to about 600 mM. The concentration of the salt (e.g., sodium chloride) in the elution composition can be about 400 mM to about 600 mM. The concentration of the salt (e.g., sodium chloride) in the elution composition can be about 200 mM to about 500 mM. The concentration of the salt (e.g., sodium chloride) in the elution composition can be about 300 mM to about 500 mM. The concentration of the salt (e.g., sodium chloride) in the elution composition can be about 400 mM to about 500 mM. The concentration of the salt (e.g., sodium chloride) in the elution composition can be about 380 mM to about 420 mM. The concentration of the salt (e.g., sodium chloride) in the elution composition can be about 400 mM.

In some embodiments, an elution composition does not have a salt concentration of 100 mM. In some embodiments, an elution composition has a salt concentration of greater than 100 mM.

An elution composition for use in the methods provided herein can have a particular pH. The pH can be, for example, about 3.8 to about 6.5. The elution composition can have a pH of about 3.8 to about 5.5. The elution composition can have a pH of about 3.8 to about 5.0. The elution composition can have a pH of about 3.8 to about 4.7. The elution composition can have a pH of about 3.8 to about 4.4. The elution composition can have a pH of about 3.8 to about 4.2. The elution composition can have a pH of about 4.0 to about 5.0. The elution composition can have a pH of about 4.0 to about 4.7. The elution composition can have a pH of about 4.0 to about 4.4. The elution composition can have a pH of about 4.0 to about 4.2. The elution composition can have a pH of about 4.2.

In some embodiments, an elution composition does not have a pH of 6.0. In some embodiments, an elution composition has a pH of less than 6.0.

An elution composition for use in the methods provided herein can have a particular combination of salt concentration and pH. For example, the salt (e.g., sodium chloride) concentration can be about 300 mM to about 600 mM, and the pH can be about 3.8 to about 5.5. The salt (e.g., sodium chloride) concentration can be about 300 mM to about 500 mM, and the pH can be about 3.8 to about 5.0. The salt (e.g., sodium chloride) concentration can be about 380 mM to about 420 mM and the pH can be about 4.0 to about 4.4. The salt (e.g., sodium chloride) concentration can be about 400 mM ,and the pH can be about 4.2.

In some embodiments, an elution composition does not have 100 mM sodium chloride at a pH of 6.0.

As demonstrated herein, applying an elution composition provided herein (e.g., with a low pH and a high salt concentration) to a cation exchange resin provided herein containing an antibody composition provided herein with H2L2 and H2L3 antibodies or antigen binding fragments thereof can result in elution of an H2L2 composition with little to no H2L3 contamination. This is because the methods provided herein can cause the H2L3 species to consistently elute late (after the H2L2 elution peak), instead of eluting both early (along with the H2L2 elution peak) as well as late (after the H2L2 elution peak).

Thus, an H2L2 composition provided herein can comprise one or more eluted column volumes. For example, an H2L2 composition can comprise a single eluted column volume selected from column volumes 1-9. An H2L2 composition can comprise two eluted column volumes selected from column volumes 1-9 (e.g., column volumes 1 and 2 or column volumes 3 and 4). An H2L2 composition an comprise three, four, five, six, seven, eight, or nine eluted column volumes selected from column volumes 1-9. An H2L2 composition can also comprise eluted column volumes 1-9 (i.e., a pool of the first nine column volumes). An H2L2 composition provided herein can comprise eluted column volumes 1-8 (i.e., a pool of the first four column volumes.) An H2L2 composition provided herein can comprise eluted column volumes 1-7 (i.e., a pool of the first four column volumes.) An H2L2 composition provided herein can comprise eluted column volumes 1-6 (i.e., a pool of the first four column volumes.) An H2L2 composition provided herein can comprise eluted column volumes 1-5 (i.e., a pool of the first four column volumes.) An H2L2 composition provided herein can comprise eluted column volumes 1-4 (i.e., a pool of the first four column volumes.) An H2L2 composition provided herein can comprise eluted column volumes 1-3 (i.e., a pool of the first four column volumes.)

Using the methods provided herein, H2L3 species can efficiently be separated from H2L2 species in an antibody composition. For example, the methods used herein can result in the production of an H2L2 composition comprising no more than 25%, no more than 20%, no more than 15%, no more than 10%, or no more than 5% of the H2L3 species that were present in the antibody composition applied to the cation exchange resin. On the other hand, the methods used herein can result in the production of an H2L3 composition comprising at least 75%, at least 80%, at least 85%, at least 90%, or at least 95% of the H2L3 species that were present in the antibody composition applied to the cation exchange resin.

By using the methods provided herein, an H2L2 composition can be obtained in which no more than 2%, no more than 1%, or no more than 0.5% of the antibodies or antigen binding fragments are H2L3 species. By using the methods provided herein, an H2L2 composition can be obtained in which at least 98%, at least 99%, or at least 99.5% of the antibodies or antigen binding fragments thereof in the H2L2 composition are H2L2 antibodies or antigen-binding fragments thereof.

By using the methods provided herein, an H2L2 composition that contains less aggregates than the antibody composition applied to the cation exchange resin can also be obtained. For example, using the methods provided herein, an H2L2 composition comprising no more than 1% aggregates or no more than 0.5% aggregates can be obtained. Using the methods provided herein, an H2L2 composition comprising about 0.3% aggregates, about 0.2% aggregates, or about 0.1% aggregates can be obtained.

Advantageously, the methods provided herein also provide a high yield. For example, using the methods provided herein an H2L2 composition that contains at least 40%, at least 45%, at least 50%, or at least 55% of the H2L2 antibodies or antigen-binding fragments thereof in the antibody composition applied to the cation exchange resin can be obtained.

In order to equilibrate the cation exchange resin and promote H2L2 and H2L3 antibody binding to the resin, an equilibration composition (or binding composition) can be applied to the resin before the antibody composition is applied to the resin. The equilibration composition (binding composition) can be used to maintain the pH and/or conductivity of the resin. Suitable buffers that can be used for this purpose are well known in the art, and include any buffer at pH that is compatible with the selected resin used in the chromatography step for separating the H2L3 and H2L2 species. The equilibration composition (binding composition) can contain, e.g., sodium acetate, in a concentration that is high enough to maintain the pH, but not too high to prevent binding of the antibodies and antigen-binding fragments thereof in an antibody composition to the cation exchange resin.

The equilibration composition (binding composition) can comprise, for example, 10 mM to 150 mM sodium acetate. The equilibration composition (binding composition) can comprise, for example, 25 mM to 150 mM sodium acetate. The equilibration composition (binding composition) can comprise 50 mM sodium acetate.

In some embodiments, the equilibration composition (binding composition) does not contain 20 mM sodium acetate. In some embodiments, the equilibration composition (binding composition) contains more than 20 mM sodium acetate.

The equilibration composition (binding composition) can also have a particular pH, e.g., about 3.8 to about 6.5. The equilibration composition (binding composition) can have a pH of about 3.8 to about 5.5. The equilibration composition (binding composition) can have a pH of about 3.8 to about 5.0. The equilibration composition (binding composition) can have a pH of about 3.8 to about 4.7. The equilibration composition (binding composition) can have a pH of about 3.8 to about 4.4. The equilibration composition (binding composition) can have a pH of about 3.8 to about 4.2. The equilibration composition (binding composition) can have a pH of about 4.0 to about 5.0. The equilibration composition (binding composition) can have a pH of about 4.0 to about 4.7. The equilibration composition (binding composition) can have a pH of about 4.0 to about 4.4. The equilibration composition (binding composition) can have a pH of about 4.0 to about 4.2. The equilibration composition (binding composition) can have a pH of about 4.2.

In some embodiments, the equilibration composition (binding composition) does not have a pH of 6.0 In some embodiments, the equilibration composition (binding composition) has a pH of less than 6.0.

As demonstrated herein, a method of separating H2L3 antibodies or antigen-binding fragments thereof from an antibody composition comprising H2L3 antibodies or antigen-binding fragments thereof and H2L2 antibodies or antigen-binding fragments thereof, can comprise (i) applying the antibody composition to a cation exchange resin so that H2L3 antibodies or antigen-binding fragments thereof and H2L2 antibodies or antigen-binding fragments thereof bind to the resin; (ii) applying an elution composition to the cation exchange resin; and (iii) collecting an H2L2 composition eluted from the resin. The method may optionally comprise applying an equilibration composition (binding composition) to the cation exchange resin before the antibody composition is applied to the cation exchange resin. As demonstrated herein, the selection of cation exchange resin as well as the pH and salt concentration of the elution composition can advantageously cause all of the H2L3 species to elute after the peak elution of H2L2 and allow for collection of an H2L2 composition with little (e.g., less than 1%) to no H2L3 species.

### VI. Uses of H2L2 Compositions

According to the methods provided herein triple-light chain (H2L3) antibodies and antigen-binding fragments thereof can be separated from double-light chain (H2L2) antibodies and antigen-binding fragments thereof to produce H2L2 compositions. Such H2L2 compositions are useful, for example, for therapeutic purposes. For example, H2L2 compositions can be used to formulate pharmaceutical compositions comprising highly pure H2L2 antibodies or antigen-binding fragments thereof, e.g., compositions comprising no more than, e.g., 1% or 0.5% H2L3 species.

H2L2 compositions produced according to the methods provided herein can also be used to produce immunoconjugates. Advantageously, the immunoconjugates produced from the H2L2 compositions provided herein will have little to no H2L3 species. Such immunoconjugates can be prepared by using a linking group in order to link a drug or prodrug to the antibody or antigen-binding fragment there. Suitable linking groups are well known in the art and include, for example, disulfide groups, thioether groups, acid labile groups, photolabile groups, peptidase labile groups and esterase labile groups. An individual immunoconjugate can contain, for example, 1, 2, 3, 4, 5, 6, 7, 8, 9, or 10 drugs or prodrugs per antibody or antigen-binding fragment thereof. A composition comprising such immunoconjugates can have an average of about 1 to about 10, about 1 to about 5, about 1 to about 3, or about 1.5 to about 2.1 drugs or prodrugs per antibody or antigen-binding fragment thereof.

An immunoconjugate composition produced according to the methods provided herein can comprise no more than 2%, no more than 1%, or no more than 0.5% of H2L3 species. An immunoconjugate composition produced according to the methods provided herein can comprise at least 98%, at least 99%, or at least 99.5% of H2L2 species.

By way of example, an H2L2 composition comprising H2L2 anti-CD123 antibodies or antigen-binding fragments thereof (e.g., huCD123-6Gv4.7; G4723A) can be conjugated to a cytotoxic agent to form an immunoconjugate. The cytotoxic agent can be, for example, an indolino-benzodiazepine cancer-killing agent, such as DGN549-C. Methods of producing such immunoconjugates provided, for example, in WO 2017/004025 and WO 2017/004026; the contents of which are entirely incorporated herein by reference.

In some embodiments, an H2L2 composition comprising H2L2 anti-CD123 antibodies or antigen-binding fragments thereof (e.g., huCD123-6Gv4.7; G4723A) can be conjugated to DGN549-C. The resulting composition of immunoconjugates can contain an average of about 1.5 to about 2.1 cytotoxins (DGN549-C) per antibody (huCD123-6Gv4.7; G4723A). The immunoconjugate composition can be formulated in sodium bisulfate to form IMGN632 as shown in FIGS. 8A (and 8B).

### EXAMPLES

It is understood that the examples and embodiments described herein are for illustrative purposes only and that various modifications or changes in light thereof will be suggested to persons skilled in the art and are to be included within the spirit and purview of this application.

### Example 1

### Assessment of H2L3 Levels

It has been previously reported that mammalian cells stably transfected to express cysteine engineered antibodies also secrete a high molecular weight species known as the triple light chain (H2L3) antibody (Gomez et al., Biotechnol Bioeng. 2010 Mar. 1; 105(4):748-60). These antibodies contain an extra (third) light chain associated with the engineered cysteine on the antibody. The separation of this new high molecular weight (HMW) species generates a challenge during downstream purification of cysteine engineered monoclonal antibodies (CysmAb), due to the similarities to the monomer species. In order to develop a robust and effective purification strategy to remove H2L3 species, the H2L3 content in an exemplary antibody was evaluated and various methods for removing the H2L3 were assessed.

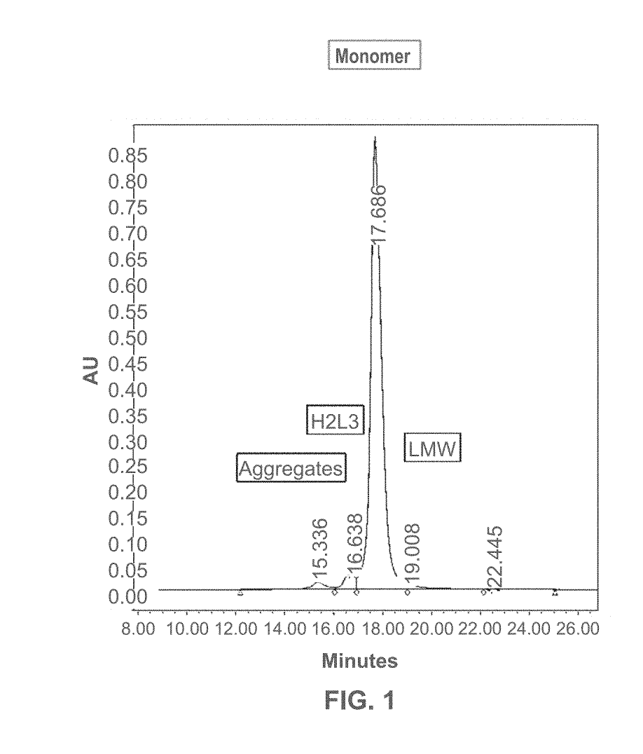

The exemplary monoclonal antibody studied was the cysteine engineered huCD123-6Gv4.7 (G4723A) antibody (see WO 2017/004025 and WO 2017/004026, the contents of which are entirely incorporated herein by reference; see also Tables 1-3 above). The antibody was expressed in CHO cells, and then it was purified from cleared cell culture supernatants using Protein A chromatography. After the Protein A chromatography, the purity (including the percentage of monomer, aggregates, H2L3, and low molecular weight species) of the antibody was assessed using the size-exclusion ultra-performance liquid chromatography (SEC-UPLC). SEC-UPLC analysis showed a H2L3 species (FIG. 1). SEC-UPLC chromatogram shows the retention time for each peak of CysmAb composition species: monomer (17.686 minutes), aggregates (15.336 minutes), H2L3 (16.638 minutes), and low molecular weight species (19.008 and 22.445 minutes) (FIG. 1).

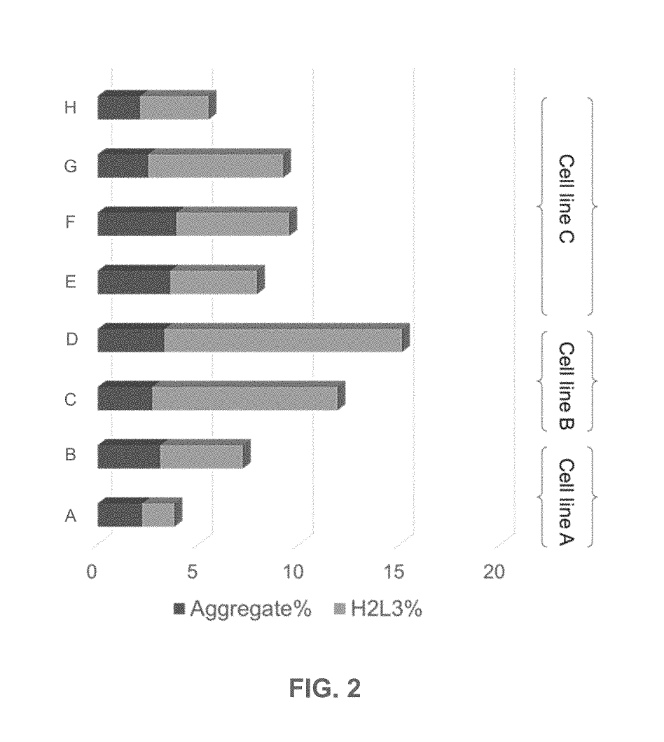

Experiments using eight different bioreactor production batches of the same antibody (A, B, C, D, E, F, G, and H) were analyzed. The amounts of H2L3 were affected by both cell line and culture condition and varied from 2-11% (FIG. 2).

### Example 2

### Ceramic Hydroxyapatite Chromatography is Inefficient for H2L3 Separation

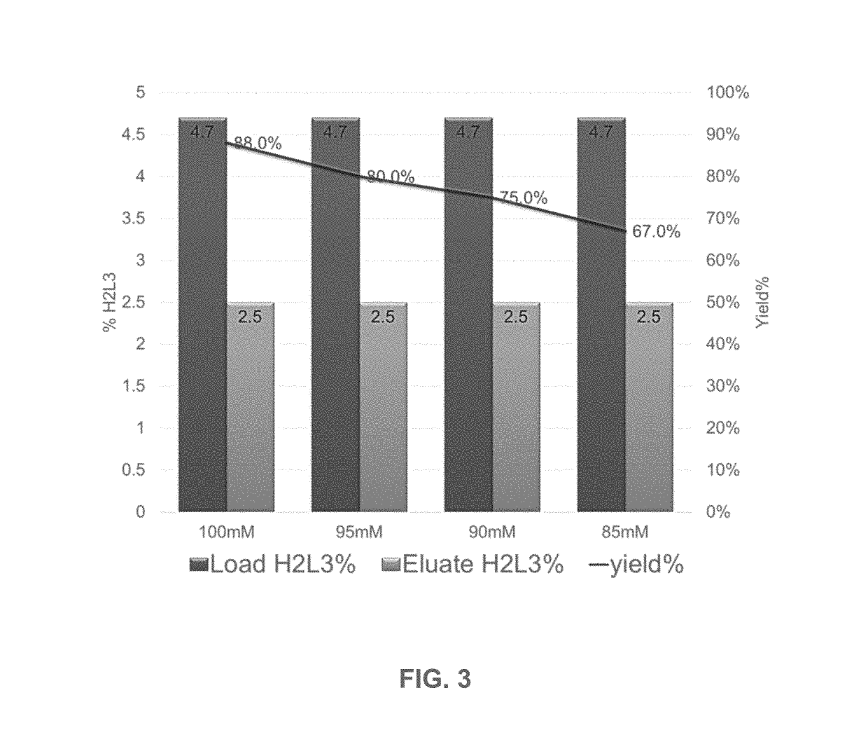

Removal of the H2L3 species was not efficiently achieved using ceramic hydroxyapatite (CHT) chromatography. (FIG. 3.) In these experiments, a CHT column was equilibrated with buffer containing 20 mM potassium phosphate at pH 6.7. CysmAb with 4.7% H2L3 species was loaded onto a CHT™ resin (BioRad Laboratories, Hercules, Calif.) and eluted by step elution using 100 mM, 95 mM, 90 mM, or 85 mM potassium phosphate buffer at pH 6.7. The elution peak was collected above 50 mAU in 1 CV fractions. The antibody step yield and H2L3% of each fraction were analyzed and used for calculation and comparison with the step yield and H2L3% of a 6 column volume (CV) virtual pool. It was thought that the high molecular weight species (i.e., aggregates and H2L3) would bind stronger on the CHT resin due to their larger size and would be eluted in later fractions. Moreover, it was thought that the elution with lower phosphate concentration would result in broader elution peaks and lower step yield of the virtual pool while improving the separation. However, FIG. 3 shows that removal of the H2L3 species was not improved across the tested phosphate range and remained as high as 2.5%. Additional experiments varying the salt concentration also failed to efficiently separate H2L3 species, and there is little room to change the pH in a CHT column. Thus, CHT chromatography was not able to efficiently separate H2L3.

### Example 3

### Optimized Cation Exchange Chromatography Efficiently Separates H2L3

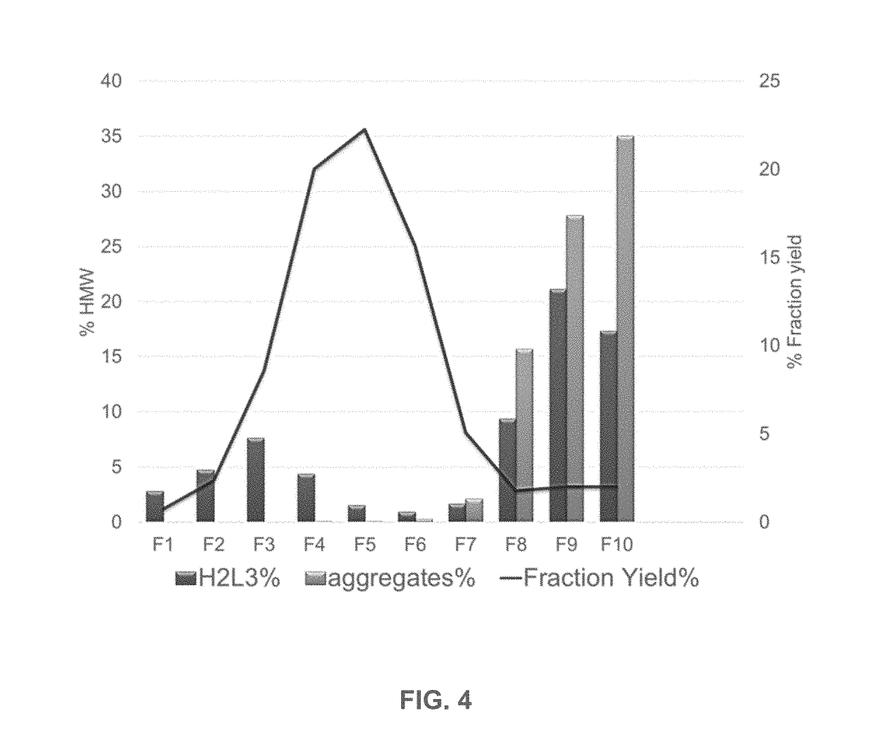

Removal of the H2L3 species was tested using POROS™ XS strong cation exchange chromatography. POROS™ strong cation exchange resin XS (Life Technologies Corporation, Carlsbad, Calif.) was equilibrated with buffer containing 50 mM sodium acetate at pH 5.5. CysmAb composition with 3% aggregates and 4% H2L3 species was loaded onto the POROS™ XS column and eluted by gradient elution using 0-200 mM NaCl over 20 CV. The elution peak was collected above 50 mAU in 0.5 CV fractions. Antibody step yield, aggregate%, and H2L3% of each fraction were analyzed and plotted against the fraction number. FIG. 4 shows that aggregate species were eluted in the late fractions (F8-F10) and efficiently separated from the major peak of eluted antibody. The H2L3 species were also eluted in different fractions than the major peak of eluted antibody. A significant portion of the H2L3 species eluted in the late fractions and were efficiently separated from the major peak of the H2L2 antibody (FIG. 4). However, a part of H2L3 species co-eluted with the earlier fractions of the antibody peak (FIG. 4). It is believe that the early-eluting H2L3 species results from the free cysteine in the H2L3 antibodies being capped with glutathione. The glutathione-capped species is more acidic (lower PI) and therefore elutes in an earlier fraction. The late-eluting H2L3 species is believed to result from the free cysteine in the H2L3 antibodies being capped with cysteine. The cysteine-capped species is less acidic (higher PI) and therefore elutes in a later fraction.

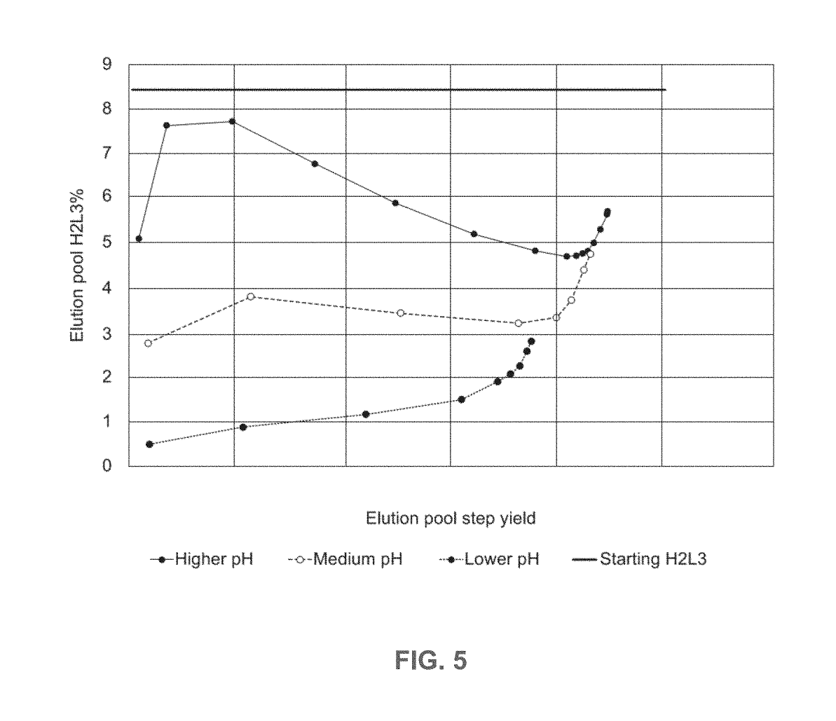

In view of these results, further optimizations of the POROS™ XS column binding and elution conditions were designed to cause the early-eluting H2L3 species to elute in a later fraction so that it would be more efficiently separated from the major peak of the H2L2 species. It was found that reducing the binding and elution pH of the Poros XS chromatography significantly improved the removal of the H2L3 species (FIG. 5).

A NaCl gradient elution was performed using POROS™ XS chromatography at pH 5.0, 4.7, and 4.4 (at pH 5.0, 150-300 mM NaCl over 20 CV; at pH 4.7 and 4.4, 0-500 mM NaCl over 25 CV). The POROS™ XS resin loading material (antibody composition) had approximately 8.4% of H2L3 species. The elution peak was collected above 100 mAU in 0.5 CV fractions. Antibody step yield and H2L3% of each fraction were analyzed. The H2L3% of a virtual pool with different collection volume was plotted against the yield of each virtual pool. Co-eluted H2L3 species in earlier fractions of the antibody peak decreased with decreasing pH. FIG. 5 shows that reducing the binding and elution pH of the POROS™ XS column significantly improved the removal of the H2L3 species that elution with a lower pH generated a product with lower H2L3 species. At pH 4.4 the H2L3 was separated efficiently from the antibody and only eluted in later fractions. Elution with a lower pH generated a product (H2L2 composition) with lower H2L3 species.

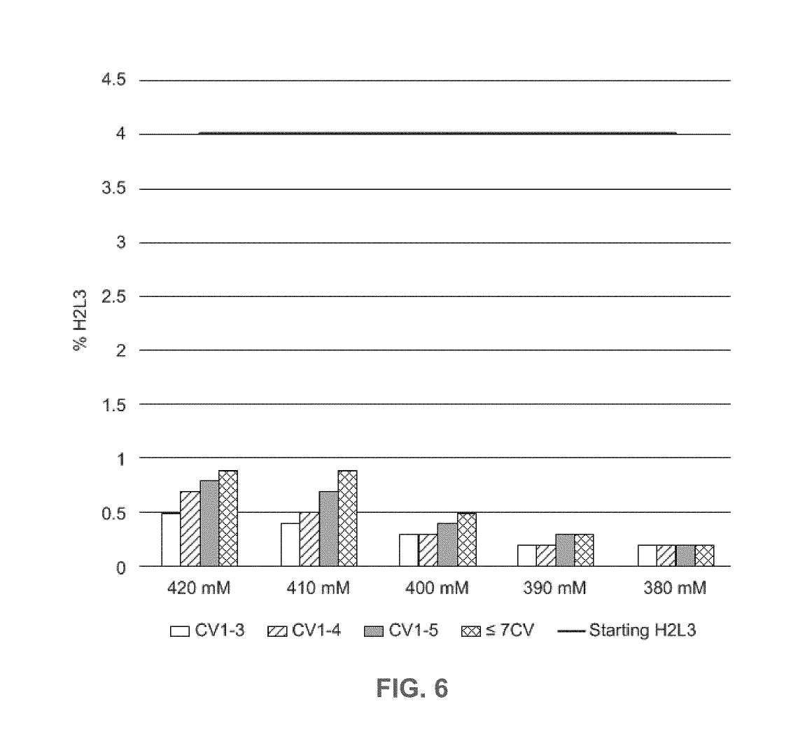

Salt concentration and collection volume of elution peak were also shown to affect the H2L3 removal (FIG. 6). In these experiments, a starting material (antibody composition) with approximately 4.1% H2L3 species was loaded onto the POROS™ XS column equilibrated with 50 mM sodium acetate at pH 4.2. Bound antibody was eluted with 380 mM to 420 mM NaCl in 50 mM Sodium Acetate buffer at pH 4.2. Elution peak was collected at above 100 mAU in 1 CV fractions. The H2L3% of virtual pools eluted at different NaCl concentration and with different collection volumes were compared. FIG. 6 shows that the lower the NaCl concentration and the less the collection volume, the lower the H2L3% was achieved in the elution pool. Across the range of tested NaCl concentration, H2L3% of virtual pools were all reduced from 4.1% to <1%.

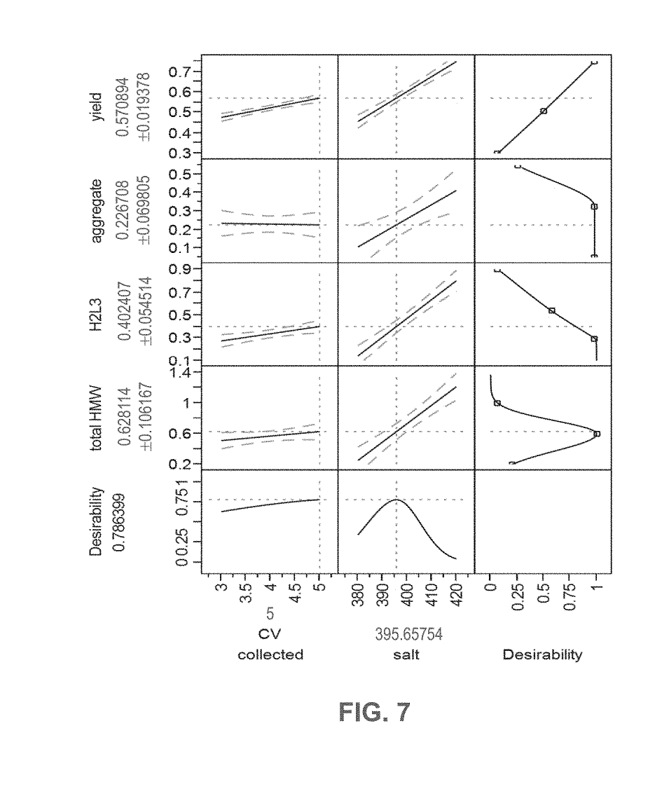

The results of the experiment in FIG. 6 were analyzed using JMP® prediction profiling tool. A mathematical model was built to predict the yield, aggregate%, H2L3%, and total high molecular weight (HMW) % under different salt concentrations and collection volumes. FIG. 7 shows that salt concentration significantly affected all the responses, while collection volume only significantly affected the step yield. The desirability of each response was set based on product quality requirement and practical considerations. The combinations of salt concentration and collection volume with highest overall desirability were considered for the final process condition.

By optimizing the binding and elution conditions including pH, salt concentration, loading density, and collection volume, the H2L3 level was consistently reduced to <1% in final product (Table 4; experiments were run in the same POROS conditions as described above, using pH 4.2, 400 mM NaCl, collecting 4 CV (i.e., column volume 1-4)).

TABLE 4
Optimization of the binding and elution
conditions including pH, salt concentration,
loading density, and collection volume.

| Experiment | A | B | C | D |
|---|---|---|---|---|
| Column size (mL) | 21 | 23 | 107 | 13800 |
| Load density (g/L resin) | 40 | 43 | 40 | 32 |
| Load aggregates % | 3% | 5% | 5% | 4% |
| Load H2L3 % | 11% | 5% | 6% | 6% |
| Eluate aggregates | 0.1% | 0.2% | 0.1% | 0.3% |
| Eluate H2L3 % | 0.8% | 0.4% | 0.6% | 0.5% |
| Step yield % | 52% | 59% | 57% | 55% |

Based on the experiments described above, it was determined that the ideal range for the pH, salt, loading density, and collection volume are as follows: pH from about 3.8 to 6.5, salt concentration from about 100 mM to 600 mM, loading density from about 10-100 g/L, and collection volume from about 1-9 CV.

It is to be appreciated that the Detailed Description section, and not the Summary and Abstract sections, is intended to be used to interpret the claims. The Summary and Abstract sections sets forth one or more, but not all, exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

1. A method of separating H2L3 antibodies or antigen-binding fragments thereof from an antibody composition comprising H2L3 antibodies or antigen-binding fragments thereof and H2L2 antibodies or antigen-binding fragments thereof, the method comprising:
(i) applying the antibody composition to a cation exchange resin so that H2L3 antibodies or antigen-binding fragments thereof and H2L2 antibodies or antigen-binding fragments thereof bind to the resin;
(ii) applying an elution composition with a pH of about 3.8 to about 5.0 to the cation exchange resin; and
(iii) collecting an H2L2 composition eluted from the resin.

2. A method of separating H2L3 antibodies or antigen-binding fragments thereof from an antibody composition comprising H2L3 antibodies or antigen-binding fragments thereof and H2L2 antibodies or antigen-binding fragments thereof, the method comprising:
(i) applying the antibody composition to a cation exchange resin so that H2L3 antibodies or antigen-binding fragments thereof and H2L2 antibodies or antigen-binding fragments thereof bind to the resin;
(ii) applying an elution composition with a salt concentration of about 300 mM to about 600 mM to the cation exchange resin; and
(iii) collecting an H2L2 composition eluted from the resin.

3. The method of claim 1, wherein no more than 2%, no more than 1%, or no more than 0.5% of the antibodies or antigen binding fragments thereof in the H2L2 composition are H2L3 antibodies or antigen-binding fragments thereof.

4.-5. (canceled)

6. The method of claim 1, wherein at least 98%, at least 99%, or at least 99.5% of the antibodies or antigen binding fragments thereof in the H2L2 composition are H2L2 antibodies or antigen-binding fragments thereof.

7. The method of claim 1, wherein the H2L2 composition comprises no more than 25%, no more than 20%, no more than 15%, no more than 10%, or no more than 5% of the H2L3 antibodies or antigen-binding fragments thereof in the antibody composition applied to the cation exchange resin.

8. The method of claim 1, wherein the H2L2 composition comprises one or more eluted column volumes selected from column volumes 1-9.

9. (canceled)

10. The method of claim 1, wherein the cation exchange resin comprises crosslinked poly(styrene divinylbenzene) and/or a suflopropyl (—CH₂CH₂CH₂SO₃—) surface functionality.

11. (canceled)

12. The method of claim 1, wherein the particle size of the cation exchange resin is about 50 μm.

13. The method of claim 1, wherein the cation exchange resin has a bimodal pore size distribution which comprises pores about 500 nM in diameter and pores about 22 nM in diameter.

14.-15. (canceled)

16. The method of claim 1, wherein the elution composition comprises a salt.

17.-21. (canceled)

22. The method of claim 2, wherein the elution composition has a pH of about 3.8 to about 6.5.

23.-24. (canceled)

25. The method of claim 1, wherein the method comprises applying an equilibration composition to the cation exchange resin prior to applying the antibody composition to the cation exchange resin.

26. The method of claim 25, wherein the equilibration composition comprises sodium acetate.

27.-28. (canceled)

29. The method of claim 25, wherein the equilibration composition has a pH of about 3.8 to about 6.5.

30. (canceled)

31. The method of claim 1, wherein the antibody composition comprises from about 10 to about 100 g/L protein.

32.-33. (canceled)

34. The method of claim 1, wherein the antibody composition has a pH of about 3.8 to about 6.5.

35. (canceled)

36. The method of claim 1, wherein about 1% to about 20% of the antibodies or antigen-binding fragments thereof in the antibody composition are H2L3 antibodies or antigen-binding fragments thereof.

37. (canceled)

38. The method of claim 1, wherein the H2L2 composition comprises at least 40%, at least 45%, at least 50%, or at least 55% of the H2L2 antibodies or antigen-binding fragments thereof in the antibody composition applied to the cation exchange resin.

39. The method of claim 1, wherein the antibody composition comprises cysteine-engineered antibodies or antigen-binding fragments thereof.

40.-43. (canceled)

44. The method of claim 1, wherein the antibody composition comprises antibodies or antigen-binding fragments thereof produced from a CHO cell line.

45. The method of claim 1, further comprising conjugating the H2L2 antibodies or antigen-binding fragments thereof in the H2L2 composition to a cytotoxin to form an immunoconjugate composition.

46. An H2L2 composition produced according to the method of claim 1.

47.-49. (canceled)

50. An immunoconjugate composition produced according to the method of claim 45.

51.-53. (canceled)

54. The method of claim 1, wherein
(i) the cation exchange resin comprises crosslinked poly(styrene divinylbenzene), a suflopropyl (—CH₂CH₂CH₂SO₃—) surface functionality, a particle size of about 50 μm, and a bimodal pore size distribution comprising pores about 500 nM in diameter and pores about 22 nM in diameter;
(ii) the elution composition comprises about 300 to 600 mM of a chloride salt and a pH of about 3.8 to about 5.0;
(iii) the antibody composition comprises from about 10 to about 100 g/L protein and about 10% to about 15% of the antibodies or antigen-binding fragments thereof in the antibody composition are H2L3 antibodies or antigen-binding fragments thereof;
(iv) the H2L2 composition comprises one or more eluted column volumes selected from column volumes 1-9; and
(v) no more than 2% of the antibodies or antigen binding fragments thereof in the H2L2 composition are H2L3 antibodies or antigen-binding fragments thereof.

55. The method of claim 1, wherein
(i) the cation exchange resin comprises crosslinked poly(styrene divinylbenzene), a suflopropyl (—CH₂CH₂CH₂SO₃—) surface functionality, a particle size of about 50 μm, and a bimodal pore size distribution comprising pores about 500 nM in diameter and pores about 22 nM in diameter;
(ii) the elution composition comprises about 400 mM NaCl and a pH of about 4.2;
(iii) the antibody composition comprises from about 30 to about 50 g/L protein and about 10% to about 15% of the antibodies or antigen-binding fragments thereof in the antibody composition are H2L3 antibodies or antigen-binding fragments thereof;
(iv) the H2L2 composition comprises eluted column volumes 1-4; and
(v) no more than 1% of the antibodies or antigen binding fragments thereof in the H2L2 composition are H2L3 antibodies or antigen-binding fragments thereof.

56. A method of separating anti-CD123 H2L3 antibodies or antigen-binding fragments thereof from an anti-CD123 antibody composition comprising anti-CD123 H2L3 antibodies or antigen-binding fragments thereof and anti-CD123 H2L2 antibodies or antigen-binding fragments thereof, the method comprising:
(i) applying the anti-CD123 antibody composition to a cation exchange resin so that anti-CD123 H2L3 antibodies or antigen-binding fragments thereof and anti-CD123 H2L2 antibodies or antigen-binding fragments thereof bind to the resin;
(ii) applying an elution composition with a pH of about 3.8 to about 5.5 to the cation exchange resin;
(iii) and collecting an anti-CD123 H2L2 composition eluted from the resin,
wherein the anti-CD123 H2L3 antibodies or antigen-binding fragments thereof and the anti-CD123 H2L2 antibodies or antigen-binding fragments thereof comprise the variable heavy chain CDR1, CDR2, and CDR3 sequences of SEQ ID NOs: 5-7, respectively and the variable light chain CDR1, CDR2, and CDR3 sequences of SEQ ID NOs: 8-10, respectively.

57. A method of separating anti-CD123 H2L3 antibodies or antigen-binding fragments thereof from an anti-CD123 antibody composition comprising anti-CD123 H2L3 antibodies or antigen-binding fragments thereof and anti-CD123 H2L2 antibodies or antigen-binding fragments thereof, the method comprising:
(i) applying the anti-CD123 antibody composition to a cation exchange resin so that anti-CD123 H2L3 antibodies or antigen-binding fragments thereof and anti-CD123 H2L2 antibodies or antigen-binding fragments thereof bind to the resin;
(ii) applying an elution composition with a with a salt concentration of about 300 mM to about 600 mM to the cation exchange resin;
(iii) and collecting an anti-CD123 H2L2 composition eluted from the resin,
wherein the anti-CD123 H2L3 antibodies or antigen-binding fragments thereof and the anti-CD123 H2L2 antibodies or antigen-binding fragments thereof comprise the variable heavy chain CDR1, CDR2, and CDR3 sequences of SEQ ID NOs: 5-7, respectively and the variable light chain CDR1, CDR2, and CDR3 sequences of SEQ ID NOs: 8-10, respectively.

58. The method of claim 56, wherein the anti-CD123 antibody comprises the variable heavy chain sequence of SEQ ID NO:1 or SEQ ID NO:3 and/or the variable light chain sequence of SEQ ID NO:2 or SEQ ID NO:4.

59.-115. (canceled)

116. A composition comprising anti-CD123 antibodies or antigen-binding fragments thereof, wherein less than 1% of the anti-CD123 antibodies or antigen-binding fragments thereof are H2L3 antibodies or antigen-binding fragments thereof, and wherein the anti-CD123 antibodies or antigen-binding fragments thereof comprise the variable heavy chain CDR1, CDR2, and CDR3 sequences of SEQ ID NOs: 5-7, respectively and the variable light chain CDR1, CDR2, and CDR3 sequences of SEQ ID NOs: 8-10, respectively.

117.-122. (canceled)

123. A composition comprising anti-CD123 immunoconjugates, wherein the immunoconjugates comprise anti-CD123 antibodies or antigen-binding fragments thereof linked to DGN549-C, wherein less than 1% of the anti-CD123 antibodies or antigen-binding fragments thereof are H2L3 antibodies or antigen-binding fragments thereof, and wherein the anti-CD123 antibodies or antigen-binding fragments thereof comprise the variable heavy chain CDR1, CDR2, and CDR3 sequences of SEQ ID NOs: 5-7, respectively and the variable light chain CDR1, CDR2, and CDR3 sequences of SEQ ID NOs: 8-10, respectively.

124.-128. (canceled)

129. The composition of claim 123, wherein the immunoconjugate has the following structure:

[symbol]

130. (canceled)
